(19) United States
(12) Patent Application Publication
(10) Pub. No.: US 2026/0120883 A1
(43) Pub. Date: Apr. 30, 2026

(54) HEALTH CARE SYSTEM FOR SAFER NAVIGATION

(71) Applicant: National Cheng Kung University, Tainan City (TW)

(72) Inventors: Ming-Chyi Pai, Tainan (TW); Jing-Jy Wang, Tainan (TW); Hung-Chang Hsiao, Tainan (TW); Ray-Bing Chen, Tainan (TW)

(21) Appl. No.: 19/003,623

(22) Filed: Dec. 27, 2024

Related U.S. Application Data
(60) Provisional application No. 63/617,892, filed on Jan. 5, 2024.

(51) Int. Cl. G16H 50/30; G16H 10/60

(52) U.S. Cl. CPC G16H 50/30; G16H 10/60

(57) ABSTRACT

A health care system for safer navigation, such as people with cognitive impairment, including: a storage unit storing at least one personal information of a user, wherein the item of the personal information includes an age and a medical history; an input interface providing the user to input at least one incident happening in any time; a processing unit respectively electrically connecting with the storage unit and the input interface, wherein the processing unit evaluates a risk index based on the personal information and receives the incident input by the user to conduct an effectiveness evaluation; and an output unit electrically connecting the processing unit, wherein the output unit is used to prompt the risk index.

### CROSS REFERENCE TO RELATED APPLICATIONS

This application claims priority of U.S. Provisional Application No. 63/617,892 filed on Jan. 5, 2024 under 35 U.S. C. § 119(e), the entire contents of which are hereby incorporated by reference.

### FIELD OF THE INVENTION

This present invention relates to the field of medical care information, particularly relates to an intelligent care system and method for people with cognitive impairment.

### BACKGROUND OF THE INVENTION

The global population of dementia patients is rapidly increasing, and the trend is also evident in Taiwan, where the number of dementia patients continues to rise. This phenomenon not only raises the demand for corresponding care services but also highlights the growing challenges associated with dementia. In the early stages of dementia, patients often face spatial navigation impairments, impacting their daily activities and potentially leading to getting lost and wandering, thereby increasing the risks they face. Therefore, providing secure mobility assistance has become particularly urgent.

The development in this technological field aims to address the continuously increasing challenges posed by dementia, aiming to provide more comprehensive, personalized, and effective care assistance.

### SUMMARY OF THE INVENTION

According to the prior art, the present invention provides an intelligent care system and method for people with cognitive impairment, which can improve the quality of life for both people with cognitive impairment and their caregivers through the use of an application, reduce the occurrence of lost and missing incidents, and deliver better care, information, and support.

According to the above object, the present invention provides an intelligent care method for people with cognitive impairment, executed through a mobile computing system, comprising: receiving a personal information of a user, wherein the item of the personal information includes age and medical history; evaluating a risk index based on the personal information; recording at least one incident happening in any time; conducting an effectiveness evaluation based on the recorded incident; and adjusting the risk index according to the effectiveness evaluation.

In a preferred embodiment of the present invention, the item of the personal information further includes gender, education level, and safe distance the one can travel home.

In a preferred embodiment of the present invention, the risk index includes a low-risk index, a medium-risk index, and a high-risk index.

In a preferred embodiment of the present invention, it further includes providing a preventive plan according to the risk index.

In a preferred embodiment of the present invention, the preventive plan includes a low-risk preventive plan, a medium-risk preventive plan, and a high-risk preventive plan.

In a preferred embodiment of the present invention, the recording step further includes recording a type, a happening time, and a location of the incident.

In a preferred embodiment of the present invention, the type of incident includes mild cognitive impairment (MCI), getting lost (GL), and going missing.

In a preferred embodiment of the present invention, it further includes conducting the effectiveness evaluation based on a number of incident-free days within a given period.

According to the above object, the present invention provides a computer-readable storage medium having stored a computer program comprising instructions, wherein the computer program is executed the mentioned intelligent care method for people with cognitive impairment.

According to the above object, the present invention provides a health care system for safer navigation, such as people with cognitive impairment, comprising: a storage unit storing at least one personal information of a user, wherein the item of the personal information includes an age and a medical history; an input interface providing the user to input at least one incident happening in any time; a processing unit respectively electrically connecting with the storage unit and the input interface, wherein the processing unit evaluates a risk index based on the personal information and receives the incident input by the user to conduct an effectiveness evaluation; and an output interface electrically connecting the processing unit, wherein the output interface is used to prompt the risk index.

In a preferred embodiment of the present invention, the item of the personal information further includes gender, education level, and safe distance the one can travel home.

In a preferred embodiment of the present invention, the processing unit further includes adjusting the risk index based on the effectiveness evaluation, the storage unit stores the updated risk index, and the output interface prompts the updated risk index.

In a preferred embodiment of the present invention, the processing unit further includes providing a preventive plan according to the risk index, and the storage unit stores the preventive plan.

In a preferred embodiment of the present invention, the input unit further includes providing the user to input a type, a happening time, a happening location of the incident.

In a preferred embodiment of the present invention, the type of incident includes mild cognitive impairment (MCI), getting lost (GL), and going missing.

In a preferred embodiment of the present invention, the processing unit further includes conducting the effectiveness evaluation based on a number of incident-free days within a given period.

### BRIEF DESCRIPTION OF THE DRAWINGS

FIG. 1 is a flow diagram illustrating an embodiment of the intelligent care method for popple with cognitive impairment according to the present invention.

FIG. 2 is a system block diagram illustrating an embodiment of the health care system for safer navigation according to the present invention.

FIG. 3, FIG. 4, FIG. 5, FIG. 6, FIG. 7, FIG. 8, FIG. 9, and FIG. 10 are schematic diagrams of different display screens for executing the health care system for safer navigation according to the present invention.

### DETAILED DESCRIPTION OF THE PREFERRED EMBODIMENT

There is a need to effectively identify elderly individuals at risk of getting lost. This requires the establishment of clear and comprehensive standards to quickly and accurately identify high-risk individuals. This involves in-depth research into cognitive functions, behavioral characteristics, and relevant clinical data. Only by understanding the specific needs of each patient can more effectively care plans be implemented.

Reducing the risk of wandering is a crucial goal. This necessitates the provision of tailor-made care programs and services to meet the unique needs of different individuals. Consideration of individual differences will be a key aspect in the design of care services, encompassing factors such as patients' lifestyle habits, daily activities, and social environments. This personalized care model not only enhances the quality of life for patients but also contributes to the reduction of wandering incidents.

Establishing a platform to track and evaluate the effectiveness of care measures is a development direction in this technology field. This requires the integration of various data sources to identify which methods have the greatest impact on patients and continually optimize care strategies. Data analysis and assessment will be the core of care innovation, aiming for more precise and targeted medical care.

Simultaneously, there is also a need to address the challenges of early diagnosis of preclinical Alzheimer's disease. Providing earlier support and treatment will have a positive impact on the long-term health of patients. This requires the development of more accurate, reliable, and non-invasive diagnostic tools to identify potential signs of dementia. Additionally, effectively applying these technologies in clinical practice to improve the accuracy and feasibility of early diagnosis.

As spatial navigation impairment is frequently seen in people with dementia and may result in unexpected injury or life-threatening conditions, a DOVE APP is designed to reduce incidents of getting lost (GL) or missing. People with cognitive impairment or their caregivers were encouraged to join the DOVE APP and install it on mobile, including mild cognitive impairment (MCI), Alzheimer dementia (AD), Lewy body dementia (LB) and other dementia. Demographic, clinical status, the scenarios of GL/missing, and the consequences were collected. During the experiment, we required reporting of previous incidents and requested the users to report each new incident.

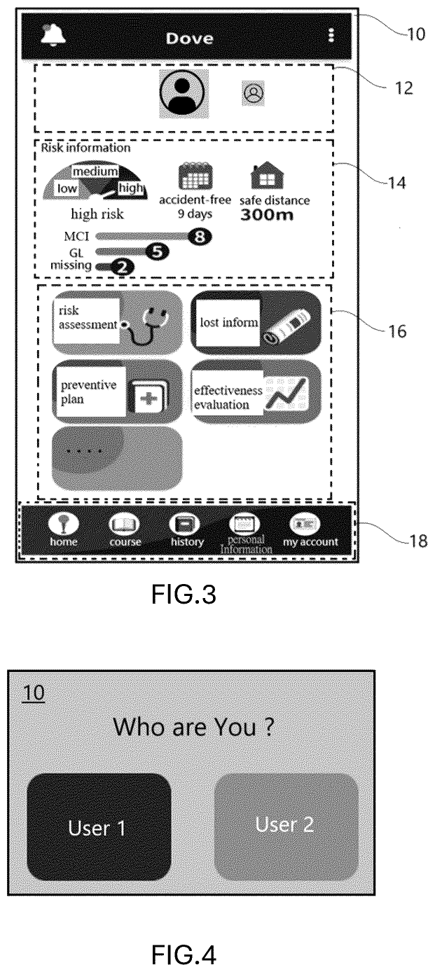

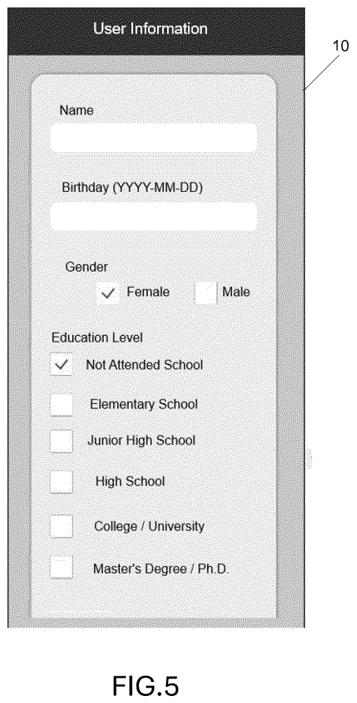

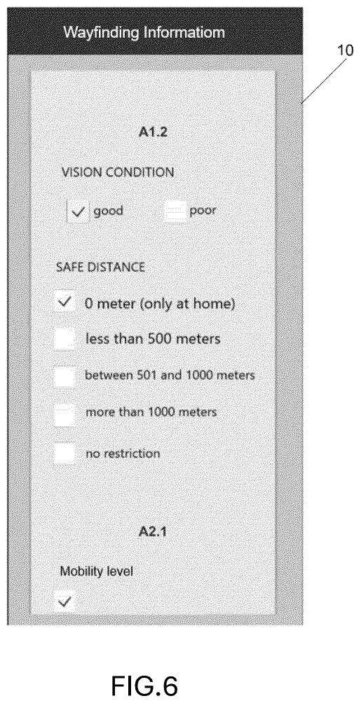

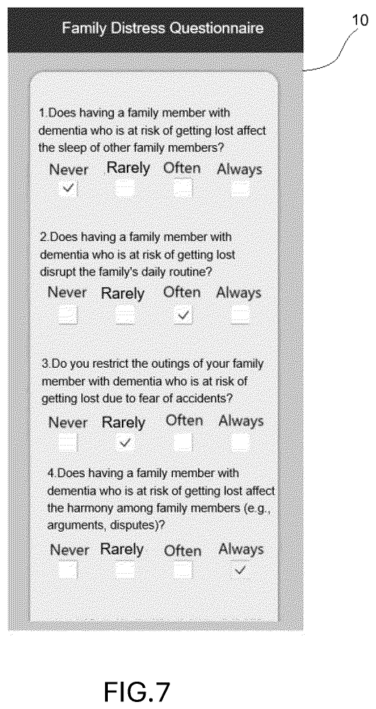

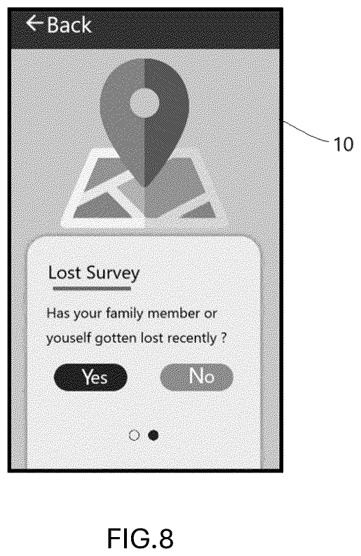

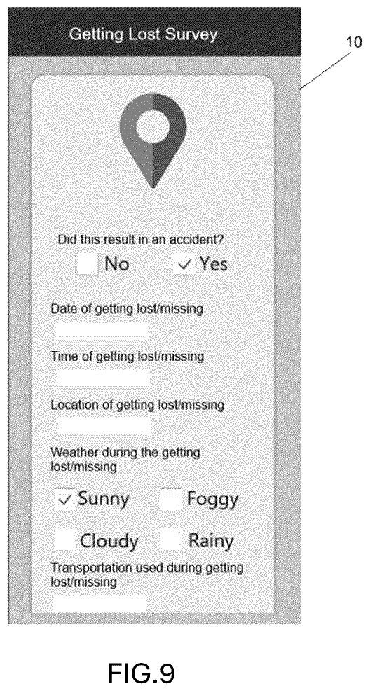

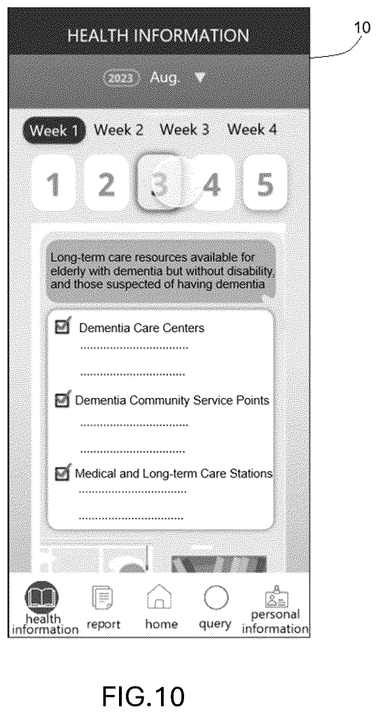

FIG. 3 displays the user interface upon entering the DOVE APP, showcasing risk information and a list of features. The risk information includes high, medium, and low risk indicators, the number of accident-free days, safe distance, obstacle event, incidents of getting lost, and instances of missing occurrences. The function list encompasses personal information, risk assessment, preventive plans, lost inform, and effectiveness evaluation. The screen also includes additional function keys such as HOME, health education courses, HISTORY, personal account, and more.

During the experiment (1 Feb. 2023 to 13 Sep. 2023), there are 488 incidents of GL or missing were uploaded to cloud databank. Among them, GL occurred more often in Clinical Dementia Rating (CDR) 0.5 group (104 vs 75) while missing more often in CDR 1.0 group (235 vs 74). Before joining the APP, the average occurrence of GL or missing of the patients were 1.54 times. The figure dropped to 0.1 after they joined the APP. Based on the individuals who have never experienced GL/missing, people diagnosed having AD with CDR 1.0 or more have an increased risk of GL (or 3.20) compared with those having non-AD and CDR 0.5 group.

The marled reduction in GL/missing after the users joined the APP may be thanks to more attention paid by caregivers. Most of the users, however, did not report through their mobile APP until been inquired by clinicians in the clinic. More efforts must be put on to improve active reporting. Safety guidelines and educational materials provided to users are expected to have beneficial effects on newly diagnosed patients.

It is an object of the present invention is to provide an intelligent care method, executed through a mobile computing system to ensure safe movements for the elder, comprising: receiving the personal information input by user, including age, gender, education level, medical history, and the distance that seniors can safely walk home on their own; determining a risk assessment level based on the personal information; providing a preventive plan according to the risk assessment level, such as low-risk plan, moderate-risk plan, and high-risk plan; reporting instances of getting lost or wandering at any time by the user's assessing; and conducting an effectiveness evaluation based on the user's report and the number of incident-free days.

It is an object of the present invention is to provide an intelligent care system for ensuring safe movements for the elder, comprising: a mobile computing system to: receive the personal information input by user, including age, gender, education level, medical history, and the distance that seniors can safely walk home on their own; determine a risk assessment level based on the personal information; provide a preventive plan according to the risk assessment level, such as low-risk, moderate-risk, and high-risk plans; report instances of getting lost or wandering at any time by the user's assessing; and conduct an effectiveness evaluation based on the user's report and the number of incident-free days.

It is an object of the present invention is to provide a non-transitory computer-readable storage medium having stored a computer program comprising instructions which, when the program is executed by a mobile computing system, cause the mobile computing system to: receive the personal information input by user, including age, gender, education level, medical history and the distance that seniors can safely walk home on their own; determine a risk assessment level based on the personal information; provide a preventive plan according to the risk assessment level, such as low-risk, moderate-risk, and high-risk plans; report instances of getting lost or wandering at any time by the user's assessing; and conduct an effectiveness evaluation based on the user's report and the number of incident-free days.

In one embodiment of the present invention, an intelligent care method, executed through a mobile computing system to ensure safe movements for the elder is provided. The method includes the following steps. Firstly, it receives personal information input from users, including age, gender, education level, medical history, and the distance that seniors can safely walk home on their own. This step establishes the necessary foundation for subsequent risk assessments.

Next, based on the personal information, a risk assessment is conducted to determine the risk level for each elderly individual. This aids in establishing a personalized care plan, offering corresponding preventive plans for low, medium, and high-risk seniors. For instance, for high-risk seniors, the high-risk plan may suggest increased companionship, more frequent monitoring, or the establishment of specific secure areas.

While implementing preventive plans, the system can also collect information on incidents of getting lost or wandering through user's assessment. This provides timely feedback, enabling caregivers to intervene promptly and ensure the safety of the elderly.

Finally, through effectiveness assessments, the system can comprehensively consider user feedback and incident-free days to evaluate the practical impact of the care plan. Conducting regular effectiveness assessments ensures the ongoing adaptability of the care plan to address changes and requirements among the elderly.

In one embodiment of the present invention, an intelligent care system for ensuring safe movements for the elder is provided. The system includes a mobile computing system. Firstly, through the mobile computing system, the system can receive user-input personal information, including age, gender, education level, and medical history, and the distance that seniors can safely walk home on their own. Simultaneously, the system establishes a comprehensive user profile.

Based on this personal information, the system conducts risk assessments, determining the risk assessment level for each elderly individual. Depending on the risk assessment level, the system provides corresponding preventive plans, such as low-risk, medium-risk, and high-risk plans. This personalized caregiving approach ensures the safety of the elderly during mobility while preserving their autonomy to the maximum extent.

In addition to preventive plans, the system continuously monitors incidents of getting lost or wandering through user's assessment. This real-time feedback enables caregivers to respond quickly and assist the elderly, ensuring they do not encounter any dangerous situations.

Finally, to continuously enhance caregiving effectiveness, the system conducts effectiveness assessments, adjusting the content of the caregiving plan based on user feedback and incident-free days. This periodic evaluation ensures the system's adaptability to changes that may occur among the elderly.

In one embodiment of the present invention, a non-transitory computer-readable storage medium is provided. The non-transitory computer-readable storage medium stores a computer program comprising instructions and the program is executed by a mobile computing system. Initially, the program enables the mobile computing system to receive user-input personal information, including age, gender, education level, medical history, and the distance that seniors can safely walk home on their own. This fundamental information establishes a comprehensive user profile for the intelligent care system.

Based on this information, the program conducts risk assessments, determining the risk assessment level for each elderly individual. According to these risk assessment levels, the program provides corresponding preventive plans, including low-risk, medium-risk, and high-risk plans. This intelligent and personalized caregiving approach ensures the safe movement of the elderly while offering adaptive support.

This intelligent caregiving system, designed to ensure the safe movement of the elderly, not only offers advanced technological support but also prioritizes delivering personalized care tailored to user needs. It enables the elderly to enjoy life in a secure and autonomous environment. Implemented through a mobile computing system, this intelligent caregiving method not only guarantees the fundamental safety of the elderly but also provides a comprehensive and personalized caregiving experience, embodying the principles of intelligent care. The intelligent and personalized caregiving approach ensures the safety of the elderly during movement while offering adaptive support.

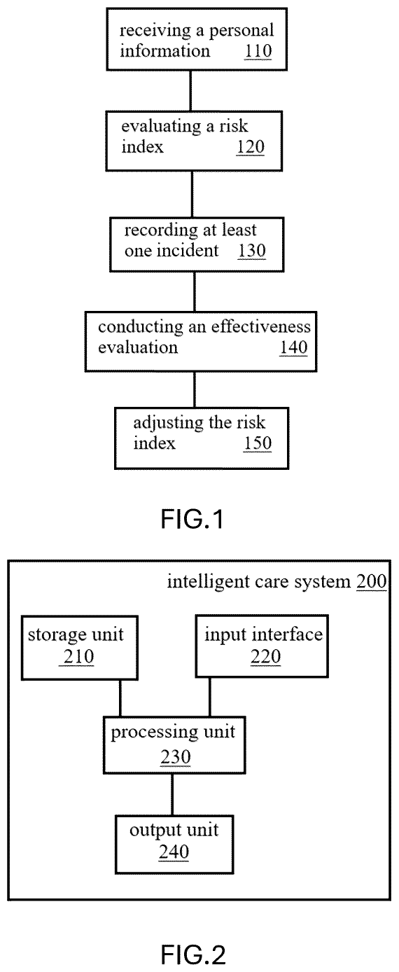

As described above, FIG. 1 illustrates a flow diagram of an embodiment of the intelligent care method for people with cognitive impairment according to the present invention. Referring to FIG. 1, the intelligent care method for people with cognitive impairment may be executed through a mobile computing system, such as a mobile device or a system including a collaborative cloud server. The intelligent care method includes the following steps. First, receiving personal information of a user (Step 110). The item of the personal information includes age and medical history. Next, evaluating a risk index based on the personal information (Step 120). Then, recording at least one incident happening in any time (Step 130). Subsequently, conducting an effectiveness evaluation based on the recorded incident (Step 140). Finally, adjusting the risk index based on the effectiveness evaluation (Step 150).

It is understood that the intelligent care method for people with cognitive impairment may be implemented in the form of a computer program containing instructions stored in one or more computer-readable storage media. The computer program, such as an application (APP), may be downloaded and executed on a mobile computing system, such as a smartphone, to perform the intelligent care method.

FIG. 2 is a block diagram illustrating an embodiment of the health care system for safer navigation. Referring to FIG. 1 and FIG. 2, the intelligent care system 200 (or health care system) for safer navigation, such as people with cognitive impairment, includes a storage unit 210, an input interface 220, a processing unit 230, and an output interface 240. The storage unit 210, such as a memory or memory module, is configured to store at least one item of personal information about the user, and the item of personal information may include age and medical history. A user may record at least one incident happening in any time through the input interface 220, such as a touch display. The processing unit 230, such as a microprocessor, ASIC, or other chips and circuits capable of computational analysis, is electrically connected to the storage unit 210 and the input interface 220. The processing unit 230 evaluates a risk index (also named as risk assessment level in paragraph [0030]) based on the personal information and receives a record related to incidents for the effectiveness evaluation. The output interface 240, electrically connected to the processing unit 230, may be a display or touch display used to prompt the risk index.

In one embodiment, the processing unit 230 also adjusts the risk index based on the effectiveness evaluation. The storage unit 210 stores the updated risk index and the output interface 240 prompts the updated risk index. In another embodiment, the processing unit 230 provides a preventive plan based on the risk index, and the storage unit 210 stores the preventive plan. Additionally, in one embodiment, the input unit 220 allows the user to input the type, the happening time, and the location of an incident.

FIG. 3 is a schematic diagram of display screen for executing the health care system for safer navigation according to the present invention. Referring to FIG. 3, in one embodiment, when the user installs and runs the intelligent care APP with a mobile device, a shown main screen 10 of the home page may include multiple zones displayed concurrently, for example but not limited to, such as a first zone 12, a second zone 14, and a third zone 16. In the first zone 12, different user options for switching and the currently logged-in user with an indication icon may be shown. Though the intelligent care system of the present invention is provided for people with cognitive impairment and their caregivers, however, anyone who concerns his/her own potential cognitive impairment may be as a user in the intelligent care system. In the second zone 14, a risk-related information may be shown, for example but not limited to, such as the risk index level of the individual with cognitive impairment, the number of days since the last incident occurred, the safe distance for returning home, and the type and count of recorded incidents, among others. In the third zone 16, there are options such as risk assessment, lost inform, effectiveness evaluation, and preventive plan provided to be selected by user. The User may view further information or proceed to the next step by clicking these options. In the fourth zone 18, there are quick accesses to key functions of the APP, such as the home page, educational courses, personal information, history review, and account management, shown to allow user to switch quickly.

Next, in one embodiment, the APP of the present invention may provide appropriate procedures of registration and identification for the user when they would like to utilize or view the message and information via the APP. FIG. 4 is a schematic diagram of display screen for executing the health care system for safer navigation according to the present invention. On the condition of completion of user registration, the user, who may be registered one with cognitive impairment, caregivers, or others, would select to be “User 1” or “User 2” in the main screen 10 shown in FIG. 4 before the main screen 10 of the home page shows. On the condition of registration procedure, the main screen 10 shown in FIG. 4 may provide a user to select who he or she is for registration and enter to next screen.

The intelligent care system of the present invention evaluates the risk index based on the entered personal information, allowing the user to complete forms by filling out, selecting, or confirming the contents. In the present invention, the item of the personal information includes age and medical history but is not limited thereto. FIG. 5, FIG. 6, and FIG. 7 are schematic diagrams of different display screens for executing the health care system for safer navigation according to the present invention. Referring to FIG. 3 and FIG. 5, when the user clicks the “personal information” icon in the fourth zone 18, the personal information of the user, including name, birthdate (age), gender, and educational background may be shown on the main screen 10 of FIG. 5 for the user to view or edit. It is understood that personal information for caregivers and patients can be entered on separate screens. Next a wayfinding survey, such as questions about the patient's vision status, safe distance (distance within which the individual can return home without getting lost), current mobility level, or other relevant questions, such as how long the patient has been unable to move freely shown on the main screen 10 of FIG. 6 may be a part of the personal information, as well as a survey for the family distress questionnaire, addressing issues like disturbances to family life, fear of restricting outings, impacts on family harmony, or feelings of helplessness and anxiety shown on the main screen 10 of FIG. 7.

Furthermore, in the present invention, the risk index is assessed based on personal information. The risk index includes low-risk level, medium-risk level, and high-risk level, corresponding to different preventive plans such as a low-risk preventive plan, medium-risk preventive plan, and high-risk preventive plan.

For example, the low-risk preventive plan focuses on raising awareness and preparedness among family members. Educating family members about dementia and the potential risk of getting lost is essential. This can be complemented by planning to reduce the likelihood of incidents, such as carrying an emergency contact card or identification documents. Additionally, optimizing the safety and familiarity of the home environment, such as enhancing visual recognition cues, can effectively prevent wandering events. Measures such as fingerprinting to establish a database in police systems for identity recognition and evaluating driving ability to ensure traffic safety can also be considered. Patients are encouraged to wear identification tools containing contact information, such as bracelets or fabric tags.

For example, the medium-risk preventive plan emphasizes maintaining cognitive function and enhancing quality of life. Patients can engage in meaningful daily activities, such as household chores or reminiscence therapy, to sustain their cognitive and social abilities. Establishing a regular daily routine, including sleep, exercise, and outdoor activities, helps improve their sense of orientation. Additionally, enhancing environmental design to strengthen orientation guidance, such as using directional signs or visual cues, can be effective. Providing safe activity spaces like enclosed gardens or walking areas ensures patients can move freely within a designated range. Family members should understand the reasons behind the patient's behaviors to meet their fundamental needs and, if necessary, reassess medications or the patient's condition.

The high-risk preventive plan focuses on strengthening safety monitoring to ensure real-time location tracking. It is recommended to use tracking devices, such as GPS watches or dedicated apps, and set up safety zone alerts while periodically checking their functionality to ensure proper operation. To prevent patients from leaving the home unintentionally, installing complex door locks or access control systems may be considered. Furthermore, the design of the home environment should be further enhanced, such as improving facilities related to balance and visual perception, thereby reducing the likelihood of the patient encountering danger due to spatial disorientation.

FIG. 8 and FIG. 9 are schematic diagrams of different display screens for executing the health care system for safer navigation according to the present invention. When a person with cognitive impairment experiences mild cognitive impairment (MCI) characterized by forgetfulness or difficulty concentrating, a temporary loss of orientation (getting lost), or wandering where they are unsure of their location or how they got there(missing), the user can record the incident either at the moment or afterward via the APP. The recorded information includes the type of incident, the time it occurred, and the location, with prompts. The APP may prompt or guide to help users document key details. Refer to FIG. 8, where a main screen 10 displays a lost survey that inquires whether the individual or their family member has recently experienced wandering. Continuing with FIG. 9, the main screen 10 displays the contents of the wandering survey questionnaire, including whether the incident resulted in any accidents (e.g., injuries), the date, time, location, weather conditions at the time, and the mode of transportation used. Additionally, the lost survey questionnaire can further inquire about the relationship between the lost/missing location and the home location (e.g., distance), the purpose of going out, and how the individual returned home after the incident.

Based on the user's request, an effectiveness evaluation can be conducted at any time after at least one incident has been recorded, such as several days, a week, or a month later. As shown in FIG. 2, the processing unit 230 may further adjust and update the risk index based on the effectiveness evaluation. The updated risk index is also stored in the storage unit 210, and the output interface 220 displays the updated risk index. The parameters used for the effectiveness evaluation may include the number and frequency of different types of incidents, the time of occurrence, the distance traveled, and the number of incident-free days within a given period. The evaluation is conducted by weighting and comparing these parameters. The risk level is adjusted according to the results of the effectiveness evaluation, and corresponding preventive plans are provided based on the different risk levels.

Please continue referring to FIG. 2 and FIG. 3, after using the intelligent care APP for a period of time, the user can select the effectiveness evaluation option to generate an interval statistical report (not shown in the figures). This interval can be set arbitrarily, such as several days, a week, a month, or a quarter. These statistical reports can either be generated simultaneously during effectiveness evaluation or accessed via a separate statistical report function button. The processing unit 230 receives the user-input incident data to generate interval statistical reports. These reports include statistics on the frequency of MCI, getting lost, or missing during different time periods, as well as trend analysis. Effectiveness evaluation can also compare past and current interval statistical reports to assess the progression of the patient's condition. These statistical reports can be stored in the storage unit and displayed by clicking the historical review button or a separately set report button. When changes in the patient's condition are detected via effectiveness evaluation (e.g., changes in the risk index), as shown in FIG. 3, the risk information in the second block 14 of the main interface 10 can be updated to provide reminders. Additionally, recorded incidents can be saved, and a report button can be designed to enable review and display.

The intelligent care system and method for people with cognitive impairment of the invention can deliver more comprehensive, personalized, and effective care assistance through the implementation of the intelligent care APP. For caregivers, the APP makes it easy to manage and record the medical history of relatives with dementia, facilitating communication with doctors during medical visits. The APP can also provide preventive plans tailored to different risk levels for caregivers to implement and offer educational course information. Referring to FIG. 3, clicking the health education course button in the fourth block 18 provides access to related information. FIG. 10 is a schematic diagram of display screen for executing the health care system for safer navigation according to the present invention. As shown in FIG. 10, the main screen 10 displays health information and resources for elderly individuals with dementia or suspected dementia who may need long-term care services. These resources include dementia shared care centers, community service stations for dementia, and neighborhood long-term care stations. This makes it easy for caregivers to obtain support information and access channels, greatly benefiting their physical and mental well-being. For people with cognitive impairment, if their condition allows them to use the APP, it can serve as a motivational tool to help them maintain social engagement and avoid social isolation. The lost inform function in the APP (as shown in the third block of FIG. 3) can also be configured to notify caregivers or establish links with police systems, such as automatic dialing. It can also display pop-up guidance messages, such as “Please stay where you are” or “Press the communication link button.” For healthcare professionals, the intelligent care APP alleviates much of the work involved in providing health and safety education to patients and caregivers. In addition to allowing real-time effectiveness evaluations, the APP serves as a detailed record of the patient's condition during medical visits, enabling more accurate diagnoses and treatment plans. For the general public, anyone with concerns about potential cognitive impairment can use the application for self-assessment, alleviating doubts or identifying early signs of the condition.

The present invention provides an intelligent care system and method designed specifically for people with cognitive impairment, enabling effective assessment of the patient's risk index and providing personalized care plans. The system, which can be implemented on a mobile device or cloud server, includes a storage unit, input interface, processing unit, and output unit. It evaluates the risk index based on the user's personal information and records incidents through the input interface for effectiveness evaluation. Based on the evaluation results, the system adjusts the risk index and provides corresponding preventive plans for different risk levels. This system not only offers effective prevention and care for patients but also helps caregivers manage the patient's medical history and access health education resources. Additionally, the system enables interval statistical reports and effectiveness evaluations, assisting caregivers in better understanding the patient's condition and adjusting care strategies as needed.

The above description is merely exemplary of the preferred embodiments of the present invention and should not be construed as limiting the scope of the invention. Any modifications, equivalents, substitutions, or improvements made within the spirit and principles of the present invention should be encompassed within the scope of protection of the invention.

What is claimed is:

1. An intelligent care method for people with cognitive impairment, executed through a mobile computing system, comprising:
receiving a personal information of a user, wherein the item of the personal information includes age and medical history;
evaluating a risk index based on the personal information;
recording at least one incident happening in any time;
conducting an effectiveness evaluation based on the recorded incident; and
adjusting the risk index according to the effectiveness evaluation.

2. The intelligent care method of claim 1, wherein the item of the personal information further includes a gender, an education level, and a safe distance the one can travel home.

3. The intelligent care method of claim 1, wherein the risk index includes a low-risk index, a medium-risk index, and a high-risk index.

4. The intelligent care method of claim 1, further comprising providing a preventive plan according to the risk index.

5. The intelligent care method of claim 4, wherein the preventive plan comprises a low-risk preventive plan, a medium-risk preventive plan, and a high-risk preventive plan.

6. The intelligent care method of claim 1, wherein the recording step further comprises recording a type, a happening time, and a location of the incident.

7. The intelligent care method of claim 6, wherein the type of the incident includes mild cognitive impairment (MCI), getting lost, and going missing.

8. The intelligent care method of claim 1, further comprises conducting the effectiveness evaluation based on a number of incident-free days within a given period.

9. A health care system for safer navigation comprising:
a storage unit storing at least one personal information of a user, wherein the item of the personal information includes an age and a medical history;
an input interface providing the user to input at least one incident happening in any time;
a processing unit respectively electrically connecting with the storage unit and the input interface, wherein the processing unit evaluates a risk index based on the personal information and receives the incident input by the user to conduct an effectiveness evaluation; and
an output interface electrically connecting the processing unit, wherein the output interface is used to prompt the risk index.

10. The system of claim 9, wherein the item of the personal information further includes a gender, an education level, and a safe distance the one can travel home.

11. The system of claim 9, wherein the processing unit further comprises adjusting the risk index based on the effectiveness evaluation, the storage unit stores the updated risk index, and the output interface prompts the updated risk index.

12. The system of claim 9, wherein the processing unit further comprises providing a preventive plan according to the risk index, and the storage unit stores the preventive plan.

13. The system of claim 9, wherein the input unit further comprises providing the user to input a type, a happening time, a happening location of the incident.

14. The system of claim 13, wherein the type of the incident includes mild cognitive impairment (MCI), getting lost, and going missing.

15. The system of claim 9, wherein the processing unit further comprises conducting the effectiveness evaluation based on a number of incident-free days within a given period.

16. A computer-readable storage medium having stored a computer program comprising instructions, wherein the computer program is executed the intelligent care method of claim 1.
